(19) United States
(12) Patent Application Publication
(10) Pub. No.: US 2021/0298979 A1
(43) Pub. Date: Sep. 30, 2021

(54) Infection Control Systems and Devices

(71) Applicants: Emory University, Atlanta, GA (US); Georgia Tech Research Foundation, Atlanta, GA (US)

(72) Inventors: Adam M. Klein, Atlanta, GA (US); Russell K. Gore, Atlanta, GA (US); David W. Wright, Atlanta, GA (US); Christopher Javellana Saldana, Decatur, GA (US)

(21) Appl. No.: 17/219,035

(22) Filed: Mar. 31, 2021

Related U.S. Application Data
(60) Provisional application No. 63/003,216, filed on Mar. 31, 2020.
(60) Provisional application No. 63/005,875, filed on Apr. 6, 2020.
(60) Provisional application No. 63/044,638, filed on Jun. 26, 2020.

(51) Int. Cl. A61G 10/00; A61G 10/02

(52) U.S. Cl. CPC A61G 10/005; A61G 10/023

(57) ABSTRACT

The disclosed infection control systems and devices can be easily and quickly deployed by clinicians and staff in emergent and routine medical environments to provide adequate protection from bio-aerosolized and droplet material while allowing appropriate access to and visibility of the patient during such procedures. The infection control devices may include a base member and a frame disposed on the base member. The frame may be configured to move between a collapsed state and an expanded state with respect to the base member. The frame may include a first support member and a second support member configured to extend vertically from the base member when in the expanded state. The devices may include an enclosure member that is disposed on the frame and the base member and configured to define an enclosure above the base member. The devices may also include access member(s) configured to provide access to the enclosure.

### CROSS-REFERENCE TO RELATED APPLICATIONS

This application claims the benefit of U.S. Provisional Application No. 63/003,216 filed Mar. 31, 2020, U.S. Provisional Application No. 63/005,875 filed Apr. 6, 2020, and U.S. Provisional Application No. 63/044,638 filed Jun. 26, 2020. The entirety of each of these applications is hereby incorporated by reference for all purposes.

### BACKGROUND

Medical procedures can expose the clinicians and staff members to a patient's aerosolized airway fluids as well as other bodily fluids. For example, airway devices, such as bag valve masks, laryngeal mask airways or endotracheal tubes, used during resuscitation or airway management procedures can result in aerosolization of the patient's airway fluids. The patient may have known or unknown infections (e.g., COVID-19). Thus, these fluids resulting from emergent and routine procedures can pose a significant transmission risk to the clinicians and ancillary staff during emergent and routine medical procedures.

Currently available solutions generally cannot be easily deployed in field and contingency hospital environments. Additionally, these solutions generally do not provide adequate protection for the clinicians and staff members from aerosolized fluids while allowing contact with the patient so that the clinicians and staff members can effectively and efficiently perform airway management procedures, resuscitation procedures, as well as other medical procedures.

### SUMMARY

Thus, there is need for easily deployed devices and systems that can adequately protect the clinicians and staff members during medical procedures while providing access to the patient for those procedures.

In some embodiments, the devices may include an infection control device that includes a base member and a frame disposed on the base member. In some embodiments, a base member may include a first side member that has first end, a second end, and a length. The base member may also include a second side member that is parallel to the first side member, the first end having a first end, a second end, and a length. The base member may further include a traverse member that has a length and is disposed between the first end of the first side member and the first end of the second side member.

In some embodiments, the frame may include a first support member, and a second support member that is spaced from the first support member with respect to the base member.

In some embodiments, the infection control device may further include an enclosure member disposed on the frame and the base member. The enclosure member may be configured to define an enclosure above the base member. The infection control device may further include one or more access members disposed on or within the enclosure member and configured to provide access to the enclosure.

In some embodiments, the frame may be configured to move between a collapsed state and an expanded state with respect to the base member. When the frame is in the expanded state, the first support member and the second support member may be configured to extend vertically with respect to the base member.

In some embodiments, the base member may have a u-shape. In some embodiments, the frame may be a wire-frame.

In some embodiments, the device may include one or more stability members disposed on the first support frame and/or the second support frame. The one or more stability members may be configured to stabilize the frame in the expanded state.

In some embodiments, the one or more stability members may include a first stability member disposed between the first support member and the second support member and a second stability member disposed between the first support member and the second support member. In some embodiments, the first stability member may include a first portion and a second portion. The second stability member may include a first portion and second portion.

In some embodiments, each of the first stability member and the second stability member may be configured to move between (i) a folded state in which the respective first portion and the second portion fold with respect to each other and (ii) a locked state in which the respective first portion and the second portion couples so that the respective portion and the second portion form a straight elongated member.

In some embodiments, the first support member may be closer to the traverse member of the base member than the second support member.

In some embodiments, the one or more access members may be configured to be coupled to one or more airflow devices. The one or more airflow devices may be configured to deliver negative pressure to the enclosure. In some embodiments, the one or more air flow devices may include a containment bag configured to deliver negative pressure to the enclosure and collect containments disposed within the enclosure. In some embodiments, the one or more access members may be air-locked.

In some embodiments, the first support member may include a first side, a second side, and a traverse member therebetween. The second support member may include a first side, a second side, and a traverse member therebetween. In some embodiments, ends of the first support member and the ends of the second support member may be configured to be disposed on the base member.

In some embodiments, the traverse member of the first support member and the traverse member of the second support member may be spaced apart with respect to the base member. In some embodiments, a portion of the traverse member of the first support member and a portion of the traverse member of the second support member may be coupled.

In some embodiments, the first support and the second support member may be different sizes. In some embodiments, the first support member and the second support member may be substantially the same size.

In some embodiments, each of the traverse member of the first support member and the traverse member of the second support member may have an arch shape.

In some embodiments, each of the traverse member of the first support member and the traverse member of the second support member may have an elongated, straight shape.

In some embodiments, the traverse member of the first support member and the traverse member of the second support member may be coupled so that the frame has a triangular side view when in the expanded state.

In some embodiments, the device may include a stabilizing member disposed on the first support member so as to be closer to or further away from the traverse member of the base member. In some embodiments, the stabilizing member may be configured to stabilize the frame in the expanded state. The base member may include one or more receiving members configured to receive a portion of the stabilizing member when the frame is in the expanded state.

In some embodiments, the stabilizing member may be a bar, such as a locking bar. In some embodiments, the one or more receiving members of the base member may be groove(s), detent(s), notch(es), among others, or a combination thereof.

In some embodiments, the frame may further include a first frame traverse member disposed between one end of the traverse member of the first support member and one end of the traverse member of the second support member; and a second frame traverse member disposed between another end of the traverse member of the first support member and another end of the traverse member of the second support member. In some embodiments, the frame may have a rectangular side view when the frame is in the expanded state.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows an example of a device according to embodiments;

FIG. 2 shows another example of a device according to embodiments;

FIG. 3 shows another example of a device according to embodiments;

FIG. 4 shows another example of a device according to embodiments;

FIG. 5 shows another example of a device according to embodiments;

FIGS. 6A-6C show the views of the device shown in FIG. 5; 6A shows the device shown in FIG. 5 in the collapsed state; FIG. 6B shows the device shown in FIG. 5 after being positioned and being moved to the partially expanded state; and FIG. 6C shows the device shown in FIG. 5 in the expanded state according to embodiments;

FIGS. 7A-7D show another example of a device according to some embodiments;

FIG. 7A shows an example of the device in the collapsed state; FIG. 7B shows the device shown in FIG. 7A after being positioned and being moved to the expanded state; FIG. 7C shows the device in the expanded state; and FIG. 7D shows the device in the stabilized, expanded state according to embodiments;

FIG. 8 shows another example of a device according to embodiments;

FIG. 9 shows an enlarged view of the access member shown in FIG. 8 according to embodiments;

FIG. 10 shows an example of an access member according to embodiments;

FIG. 11 shows an example of a device having the access member shown in FIG. 10 according to embodiments;

FIG. 12 shows an example of a device according to embodiments being used in an airway management procedure;

FIG. 13 shows another example of a device according to embodiments being used in an airway management procedure;

FIGS. 14A-B show views of the device shown in FIG. 13; FIG. 14A shows an example of the device shown in FIG. 13 being readied for removal; and FIG. 14B shows an example of the device shown in FIG. 14A in a partially collapsed state;

FIGS. 15A-B show another example of a device according to embodiments;

FIG. 15A shows an example of the device in the collapsed state; and FIG. 15B shows an example of the device shown in FIG. 15A in the expanded state;

FIGS. 16A-B show another example of a device according to embodiments;

FIG. 16A shows an example of the device in the collapsed state; and FIG. 16B shows an example of the device shown in FIG. 16A in the expanded state;

FIGS. 17A-B show another example of a device according to embodiments;

FIG. 17A shows an example of the device in the collapsed state; and FIG. 17B shows an example of the device shown in FIG. 17A in the expanded state; and

FIGS. 18A-B show another example of a device according to embodiments;

FIG. 18A shows an example of the device in the collapsed state; and FIG. 18B shows an example of the device shown in FIG. 18A in the expanded state.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

In some embodiments, the disclosure may relate to infection control systems and devices that can be easily and quickly deployed by clinicians and staff in emergent and routine medical environments to provide adequate protection from bio-aerosolized and droplet material while allowing appropriate access to and visibility of the patient during such procedures. For example, the infection control devices may include a frame that may be configured to easily move between a collapsed state and an expanded state with respect to a base support/member. This way, the infection control devices may act like a tent and “pop-up” when needed.

By way of example, the infection control devices can be stored and disposed in the collapsed state. When being deployed in use, the infection control devices may be positioned in the collapsed state and then moved to its expanded state to provide a protected treatment space when needed for a medical procedure. In its deployed, expanded state, the infection control devices may be configured to define an enclosure that provides a protected space in which the clinicians and staff can safely access to the patient for a medical procedure. By providing a protected space, transmission of infectious viruses or diseases, such as COVID-19, from the patient to the clinicians and ancillary staff can be prevented during emergent and routine medical procedures. After its use, the devices may be collapsed so as to be easily discarded, for example, by sliding off the patient, while containing the patient's particulates. This way, the devices according to the disclosure can be feasible for use in multiple settings such as the field by EMS or military providers, emergency room, operating room, intensive care unit, hospital, among others, or any combination thereof.

In some embodiments, the devices may include one or more access members that is configured to provide access to the protected treatment space provided by the enclosure for the clinicians and/or staff. The one or more access members may be configured to provide direct access to the patient, to deliver medical instruments to a target area of the patient, among others, or a combination thereof. For example, the one or more access members and/or the defined enclosure may be configured to permit medical procedures, such as intubation/extubation, airway management (bag valve mask oxygenation, effective placement of an airway endotracheal tube, confirmation of tube placement, connection to a ventilator), resuscitation efforts (e.g., chest compressions), suctioning of secretions and/or contaminated contents, connection to other instruments, connection to a negative airflow device configured to remove particulates and/or minimize fogging, among others, or any combination thereof. This way, the devices and systems of the disclosure may be configured to be used in many different medical procedures including but not limited to tracheotomy, sinus/nasal surgery, ear surgery, eye surgery, flexible bronchoscopy, upper endoscopy/TNE, dental/DMFS procedures, orthopedic surgeries, other types of surgeries and medical procedures, etc.

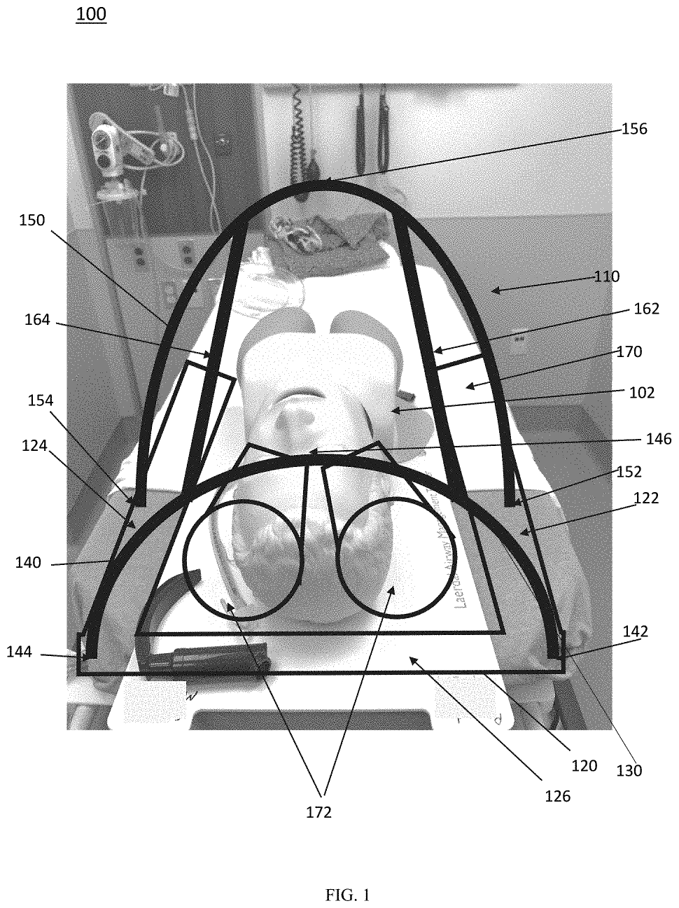

FIGS. 1-18B show examples of infection control systems and devices according to embodiments. It will be understood that the systems and devices may not be limited to the configuration and/or combination of the features of the infection control systems and devices as shown and described with respect to the figures. The devices may include any combination of the features as shown and described.

In some embodiments, the devices may include at least one base member, at least one frame disposed on the at least one base member, and an enclosure member disposed on the at least one frame and the at least one base member. In some embodiments, the at least one frame may be configured to move with respect to the base member between a collapsed state and an expanded state. In the expanded state, the enclosure member may be configured to provide an enclosure providing a protected space in which a medical procedure may be performed.

In some embodiments, the frame may include one or more support members. The one or more support members may be configured to be movable with respect to the base member between the collapsed and expanded states. The one or more support members may define the dimensions (e.g., width and/or height) of the protected space. In some embodiments, the sizes of the one or more support members, shapes of the one or more support members, and/or spacing of the one or more support members with respect to the base member and/or other support members may depend on the desired procedure/application.

In some embodiments, the one or more support members may be made of one or more semi-rigid materials (such as plastic, metal (e.g., wire-frame), among others, or a combination thereof); may be made of the material of the enclosure member, such as a reinforced seam; among others; or any combination thereof.

In some embodiments, the frame may include a first support member and second support member spaced from the first support member with respect to the base member. The first support member and the second support member may be configured to be movable with respect to the base member between a collapsed state and an expanded state. The first support member and the second support member may define the dimensions (e.g., width and/or height) of the protected space. In some embodiments, the sizes, shapes, and/or spacing of the first support member and the second support member may depend on the desired procedure/application. By way of example, the first support member and the second support member may be of the substantially the same size, the first support member may be larger than the second support member, the second support member may be larger than the first support member, among others, or a combination thereof. For example, the first support member may be larger than the second support member with respect to height; or the second support member may be larger than the first support member with respect to height.

The first support member and the second support member may have any shape. The one or more support members may have closed shape, an open shape, among others, or a combination thereof. For example, the first support member and/or the second support may have a semi-circular shape, an arch/curved shape, a quadrilateral shape (a square or rectangular shape), a trapezoidal shape, a U-shape (a curved U-shape, a polygonal (e.g., rectangular or square-like U-shape), a symmetrical U-shape, a non-symmetrical U-shape, and any combination thereof), among others, or a combination thereof.

In some embodiments, the base member may have a first side member that has first end, a second end, and a length; a second side member that is parallel to the first side member, the first end having a first end, a second end, and a length; and a traverse member that has a length and is disposed between the first end of the first side member and the first end of the second side member. The base member may end at the second end of second side member and the second end of the first side member. The first side member, the second side member, and the traverse member may form a U-shape. The base member may have a polygonal (e.g., rectangular or square-like) U-shape as shown. In some embodiments, the base member have a different u-shape, such as a curved U-shape, a symmetrical U-shape, among others, or a combination thereof. By providing an open side, the first side member, the second side member, and the traverse member may define an area in which the patient may be disposed, and enclosed by the enclosure member (protected space). For example, the patient's head, the patient's foot, a side of the patient, etc., may be disposed in the area defined by the first side member, the second side member, and the traverse member when in use.

In some embodiments, the first support member and the second support member may be coupled to each other by one or more connecting members (e.g., a mechanical connecting member(s) (e.g., pins, hinges, other mechanical fasteners, etc.), a portion of the enclosure member, other fasteners (e.g., an adhesive), among others, or a combination thereof), by itself (e.g., partially integrated (e.g., at the respective traverse members)), among others, or a combination thereof. In some embodiments, the frame may be coupled to the base member by one or more connecting members, such as one or more mechanical connecting members, a portion of the enclosure member, other fasteners, by itself, among others, or a combination thereof.

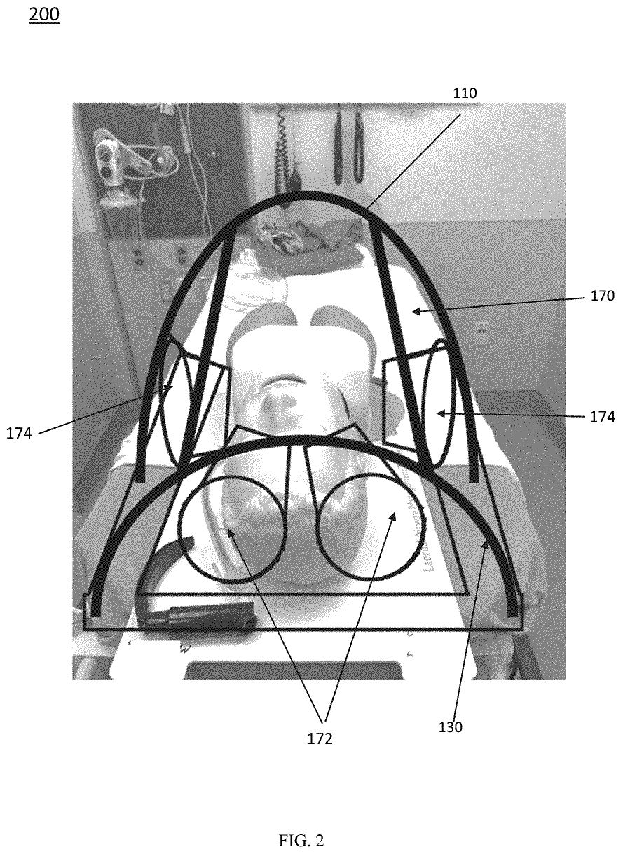

FIGS. 1 and 2 show examples 100 and 200 of a device 110 in which a frame 130 having a first support member 140 and a second support member 150 spaced apart are shown in the expanded state with respect to a patient dummy 102 according to some embodiments. In some embodiments, the device 110 may include a base member 120, a frame 130, and an enclosure member 170 disposed on the frame 130 and the base member 120. In this example, the first support member 140 and the second support member 150 may be substantially the same size.

In some embodiments, the base member 120 may include a first side member 122, a second side member 124 parallel to the first side member 122, and a traverse member 126 that is disposed between the first side member 122 and the second side member 124. As shown in FIGS. 1 and 2, the base member 120 may have a U-shape.

In some embodiments, the frame 130 may include a first support member 140 and a second support member 150. In some embodiments, the first support member 140 may include a first end/side 142, a second side/end 144, and a traverse member 146 disposed therebetween; and the second support member 150 may include a first side/end 152, a second side/end 154 and a traverse member 156 disposed therebetween. The ends of the first sides 142 and 152 may be disposed on the first side member 122 and the ends of the second sides 144 and 154 may be disposed on the second side member 124.

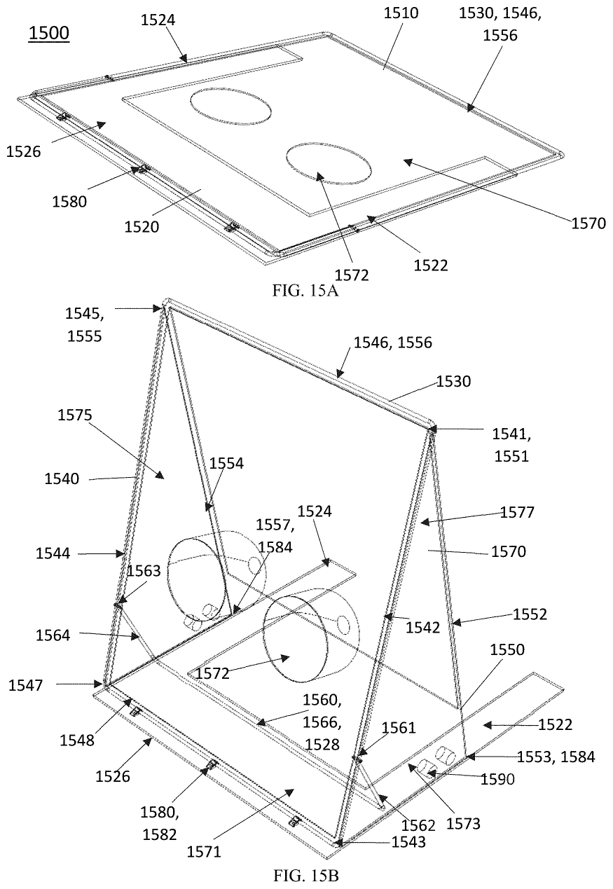

In some embodiments, the traverse member 146 and 156 may be an arch or curved as shown in FIGS. 1 and 2. In other embodiments, the traverse member 146 and 156 may have a different shape, for example, a straight, elongated shape, for example, as shown in FIGS. 15A-18B.

In some embodiments, the enclosure member 170 may be disposed on the frame 130 and the base member 120. In some embodiments, the enclosure member 170 may include a channel in which the first support member 140 and/or the second support member 150 may be disposed.

In some embodiments, the first support member 140 and the second support member 150 may be a rod. In some embodiments, the rod may be made of one or more semi-rigid materials, such as plastic, metal (e.g., wire-frame), among others, or a combination thereof. In some embodiments, the first support member 140 and the second support member 150 may be made of the material of the enclosure member 170, such as a reinforced seam.

In some embodiments, the first support member 140 and/or the second support member 150 and/or the base member 120 may be configured to be expandable. For example, the first support member 140 and/or the second support member 150 and/or the base member 120 may be a channel that is defined by the enclosure material and that is configured to be inflated.

In some embodiments, the enclosure member 170 may be disposed so as to extend past the base member 120. For example, the enclosure member 170 may be capable of covering a length of a patient's body. In some embodiments, the end (not shown) of the enclosure member 170 and/or the frame 130 may include one or more fastening members configured to removably secure the enclosure member 170 to the patient and/or the frame (e.g., the second support member 160). For example, the one or more fastening members include one or more ties, hoop and loop fasteners, additional base/frame members, among others, or any combination thereof.

In some embodiments, the enclosure member 170 may be made of a transparent material(s). For example, the enclosure member 170 may be made of one or more materials that is configured to be impermeable to aerosolized materials. For example, the one or more materials may include synthetic materials, such as nylon, plastic, among others, or a combination thereof.

In some embodiments, the device 110 may include one or more access members (or ports) disposed on or within the enclosure member 170. In some embodiments, the one or more access members may include one or more pouches or walled member configured to provide one or more sets of user's hands access to the enclosed space (enclosure) defined by the enclosure member 170; one or more pouches or walled member configured to provide one or more instruments access to the enclosed space/enclosure defined by the enclosure member 170; an adapter or a valve configured to attach to or couple to an instrument, such as ventilator tubing, ambu bag, negative pressure system, positive pressure system, among others, or any combination thereof; one or more among others, or a combination thereof. In some embodiments, one or more of the one or more access members may be configured to be sealed, such as airlocked, to prevent the movement of air into or from the enclosure defined by the enclosure member. For example, the one or more access members may include a gasket or walled structure configured to eliminate the movement of air into or from the enclosed space. See, for example, FIGS. 8-14B.

For example, as shown in FIG. 1, the device 110 may include one or more access members 172 configured to provide access to the enclosed space/enclosure. For example, the one or more access members 172 may be configured to receive a set of a user's hands. In some embodiments, the members 172 may be disposed on or within the enclosure member 170 adjacent to the traverse member 126. This way, one or more of the members 172 may be configured to provide access to the user's head, for example, for airway management procedures.

In some embodiments, the one or more access members 172 may include arm sleeves that are configured to permit access to the patient's 102 head, airway, and neck. In some embodiments, the sleeves may be tapered with a large opening narrowing to a tight sleeve to increase range of motion and minimize traction on enclosure material. In some embodiments, the arm sleeves may include an integrated glove or an adhesive material along the inside of the arm sleeve. For example, a clinician placing hands inside the sleeves wearing gloves can self-adhere the arm sleeve adhesive to form a seal.

In some embodiments, the device 110 may include additional or alternative access members. For example, the device 110 may include a second set 174 of access members disposed on the enclosure member 170 adjacent to the first side member 122 and the second support member 124. For example, these access members may each be configured to receive a set of user's hands (for example, additional staff to assist with an airway management procedure).

In some embodiments, the device 110 may include additional access members, such as ports. For example, one or more access members may be configured to couple/attach to one or more air flow devices (e.g., devices configured to deliver negative pressure), instruments, tubing, among others, or a combination thereof. See, for example, FIGS. 11-14B. The number, location, size, shape, type, configuration, etc. of the one or more access members may depend on the purpose of the infection control device.

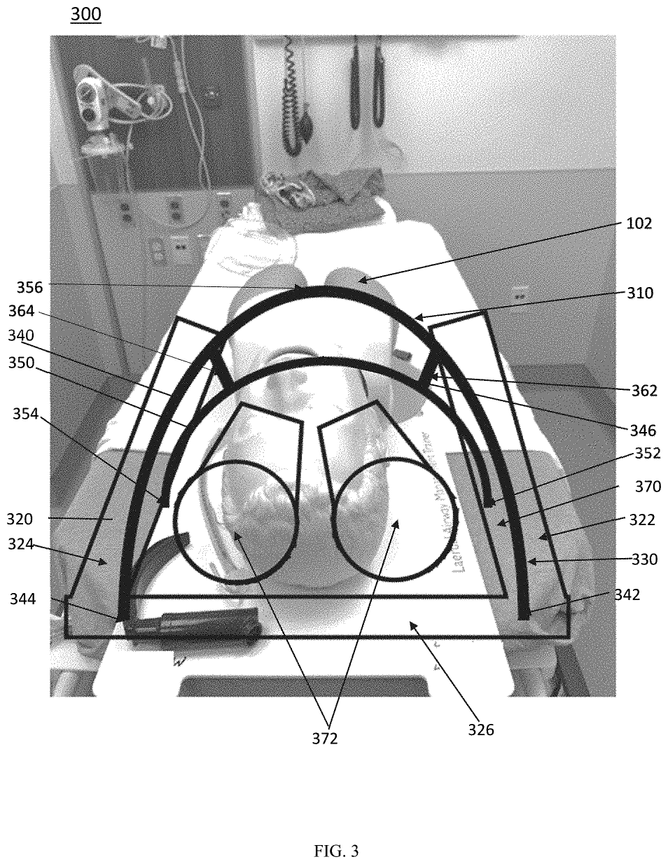

In some embodiments, the first support member and the second support member may be of different sizes. In some embodiments, the first support member may be configured to be larger than the second support member, as shown in the example 300 of FIG. 3.

Like the device 110 shown in FIGS. 1 and 2, FIG. 3 shows a device 310 having a first support member 340 and a second support member 350 spaced apart in the expanded state with respect to the patient dummy 102 according to some embodiments. In some embodiments, the device 310 may include a base member 320, a frame 330, and an enclosure member 370 disposed on the frame 330 and the base member 320. In the example 300, the first support member 340 may be larger than the second support member 350 so that the height of the traverse member (arch) 342 of the first support member 340 may be higher than the height of the traverse member (arch) 352 of the second support member 350.

In some embodiments, like the device 110, the base member 320 may have a U-shape. The base member 320 may include a first side member 322, a second side member 324 parallel to the first side member 322, and a traverse member 326 that is disposed between the first side member 322 and the second side member 324.

In some embodiments, the frame 330 may include a first support member 340 and a second support member 350. In some embodiments, the first support member 340 may include a first side/end 342, a second side/end 344, and a traverse member 346 disposed therebetween; and the second support member 350 may include a first side/end 352, a second side/end 354 and a traverse member 356 disposed therebetween. The ends of the first sides 342 and 352 may be disposed on the first side member 322 and the ends of the second sides 344 and 354 may be disposed on the second side member 324.

In some embodiments, the traverse member 346 and 356 may be an arch or curved as shown in FIG. 3. In other embodiments, the traverse member 346 and 356 may have a different shape, for example, a straight, elongated shape, for example, as shown in FIGS. 15A-18B.

In some embodiments, like the enclosure member 170 of FIG. 1, the enclosure member 370 may be disposed on the frame 330 and the base member 320. In some embodiments, like the access members 172, the device 310 may include a set of access members 372 disposed on/within the enclosure member 370 and configured to provide a set of user's hands access to the enclosed space/enclosure defined by the enclosure member 370.

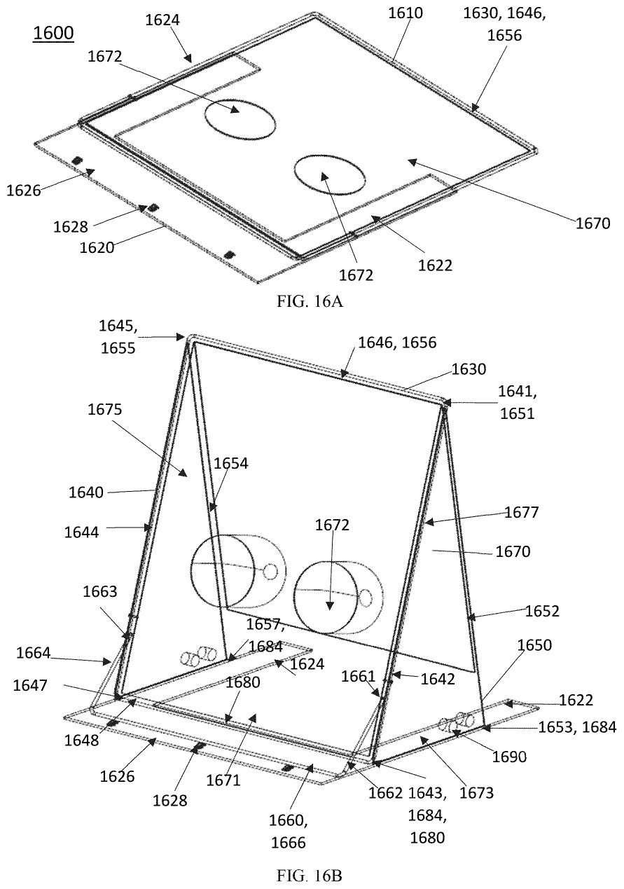

In some embodiments, the devices may further include one or more stabilizing members configured to stabilize the frame in the expanded state. In some embodiments, the one or more stabilizing members may be disposed between the first support member and the second support member. In some embodiments, the one or more stabilizing members may include a first portion, a second portion, and a hinge therebetween. This way, the frame may be removably locked in the expanded state by moving each first portion and each second portion from a folded state to a locked state so that the first portion and the second portion form an elongated member, for example, as shown in FIG. 4. In some embodiments, the one or more stabilizing members may be different, such as a locking bar, as shown in FIGS. 15A-16B.

FIG. 4 shows an example 400 of a device 410 according to some embodiments.

Like the devices of FIGS. 1-3, FIG. 4 shows a device 410 having a first support member 440 and a second support member 450 spaced apart are shown in the expanded state with respect to a patient dummy 102 according to some embodiments. In some embodiments, like the devices 110 and the 310, the device 410 may include a base member 420, a frame 430, and an enclosure member 470 disposed on the frame 430 and the base member 420.

In some embodiments, like the devices 110 and 310, the base member 420 may have a U-shape. The base member 420 may include a first side member 422, a second side member 424 parallel to the first side member 422, and a traverse member 426 that is disposed between the first side member 422 and the second side member 424.

In some embodiments, the frame 430 may include a first support member 440 and a second support member 450. In some embodiments, the first support member 440 may include a first side/end 442, a second side/end 444, and a traverse member 446 disposed therebetween; and the second support member 450 may include a first side/end 452, a second side/end 454 (not shown) and a traverse member 456 disposed therebetween. The first side/end 442 may be disposed on the first side member 422 and the second side/end 444 may be disposed on the second side member 424, and the first side/end 452 may be disposed on the first side member 422 and the second side/end 454 may be disposed on the second side member 424.

In some embodiments, the traverse member 446 and 456 may be an arch or curved as shown in FIG. 4. In other embodiments, the traverse member 446 and 456 may have a different shape, for example, a straight, elongated shape, as shown in FIGS. 15A-18B. In some embodiments, the second support member 450 may be larger than the first support member 440 so that the height of the traverse member (arch) 452 of the second support member 450 may be higher than the height of the traverse member (arch) 442 of the first support member 440, as shown in FIG. 4. This way, the enclosure space defined by the enclosure member 470 may be sized to accommodate the needed access to and visualization of the patient (e.g., patient 102).

In some embodiments, like the enclosure members of FIGS. 1-3, the enclosure member 470 may be disposed on the frame 430 and the base member 420. In some embodiments, like the access members shown in FIGS. 1-3, the device 410 may include a set of access members 472 and 474 disposed within the enclosure member 470 and configured to provide a set of user's hands access to the enclosed space/enclosure defined by the enclosure member 470.

In some embodiments, the device 410 may include a first stabilizing member 462 and a second stabilizing member 464 configured to stabilize the frame in the expanded state. In some embodiments, the one or more stabilizing members 462 and 464 may be disposed between the first support member 440 and the second support member 450. In some embodiments, the one or more stabilizing members 462, 464 may include a first portion, a second portion, and a hinge therebetween. This way, the frame 430 may be removably locked in the expanded state by moving each first portion and each second portion of the first stabilizing member 462 and the second stabilizing member 464 from a folded or collapsed state to a locked, expanded state so that the first portion and the second portion form an elongated member, for example, as shown in FIG. 4.

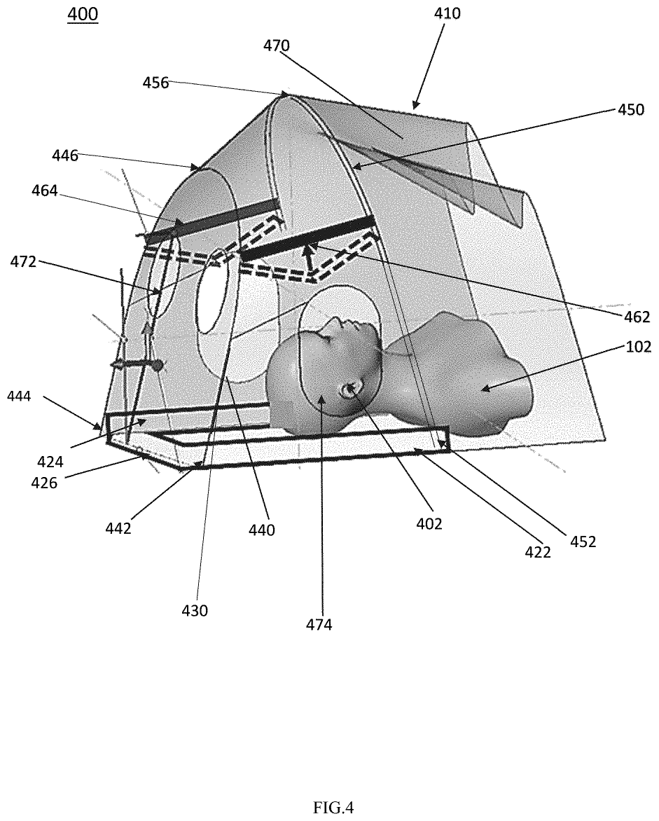

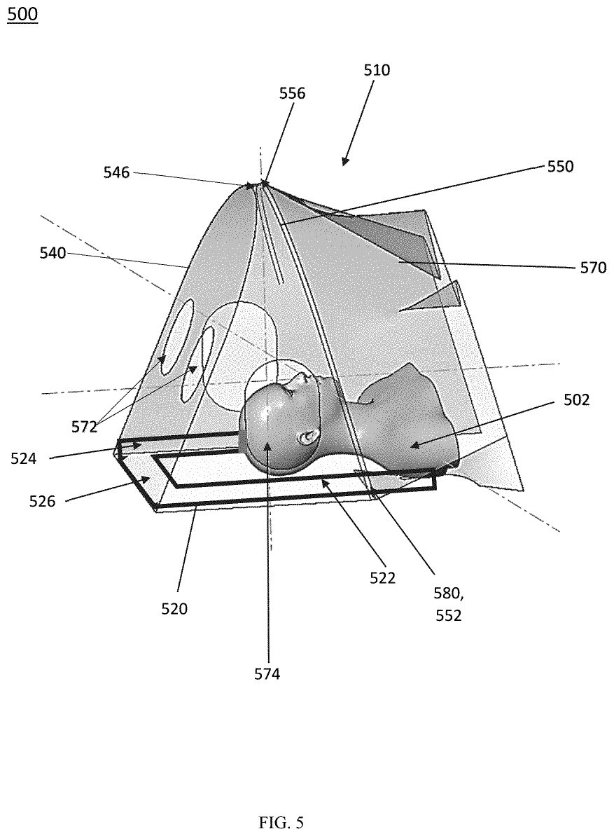

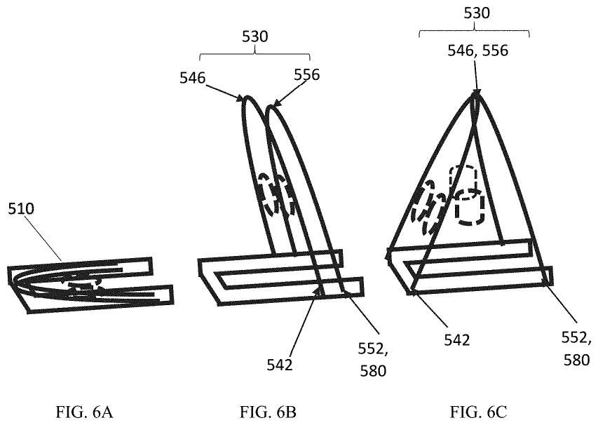

In some embodiments, the first support member and the second support member may be configured to be adjacent to each other in the expanded configuration, for example, as shown in the FIGS. 5 and 6A-C FIG. 5 shows an example 500 of a device 510 having a first support member 540 and a second support member 550 angled toward each other in the expanded state with respect to a patient dummy 102 according to some embodiments. In some embodiments, like the devices 110, 310 and 410, the device 510 may include a base member 520, a frame 530, and an enclosure member 570 disposed on the frame 530 and the base member 520.

In some embodiments, like the devices of FIGS. 1-4, the base member 520 may have a U-shape. The base member 520 may include a first side member 522, a second side member 524 parallel to the first side member 522, and a traverse member 526 that is disposed between the first side member 522 and the second side member 524.

In some embodiments, the frame 530 may include a first support member 540 and a second support member 550. In some embodiments, the first support member 540 may include a first side/end 542, a second side/end 544, and a traverse member 546 disposed therebetween; and the second support member 550 may include a first side/end 552, a second side/end 554 and a traverse member 556 disposed therebetween. The ends of the first sides 542 and 552 may be disposed on the first side member 522 and the ends of the second sides 544 and 554 may be disposed on the second side member 524.

In some embodiments, the traverse member 546 and 556 may be an arch or curved as shown in FIG. 5. In other embodiments, the traverse member 546 and 556 may have a different shape, for example, a straight, elongated shape as shown in FIGS. 15A-18B.

In some embodiments, the height of the traverse member (arch) 552 of the second support member 550 may be similar to the height of the traverse member (arch) 542 of the second support member 540.

In some embodiments, like the enclosure members of FIGS. 1-4, the enclosure member 570 may be disposed on the frame 530 and the base member 520. In some embodiments, like the access members of FIGS. 1-4, the device 510 may include a set of access members 572 and 574 disposed within the enclosure member 570 and configured to provide a set of user's hands access to the enclosed space/enclosure defined by the enclosure member 570.

FIGS. 6A-C show the device 510 moving between the collapsed state to the expanded state. FIG. 6A shows the device 510 in the collapsed state; FIG. 6B shows the device 510 after being positioned and being moved to the expanded state; and FIG. 6C shows the device 510 in the expanded state as shown in FIG. 5.

In some embodiments, one or more of the support members may be rotationally disposed with respect to the base member. In this example, the first side/end 542 and the second side/end 544 of the second support member 550 may be fixed with respect to the base member 520 by one or more connecting members. The one or more connecting members may include mechanical connecting member(s) (e.g., pins, hinges, other mechanical fasteners, etc.), a portion of the enclosure member, other fasteners (e.g., an adhesive), among others, or a combination thereof). In some embodiments, the second support member 550 may be rotationally disposed with respect to the base member 520. For example, the one or more connecting members may include a hinge 580 disposed between the base member 520 and the side/end 552 and a hinge (not shown) between the base member 520 and the side/end 554.

In some embodiments, the first support member 540 may be configured to move with respect to the base member 520 and configured to removably lock the ends/sides 542, 544 of the first support member 540 to the base member 520. For example, the base member 520 may include a locking member complimentary to the member disposed on the ends/sides 542 and 544.

For example, the base member 520 may include a track disposed along the length of the first side member 522 and the second side member 524 on which the first support member 540 may be configured to move. In some examples, the base member 520 may include one or more holes along the track in which a complimentary member (e.g., peg) disposed on the ends/sides 522 and 524 may be disposed.

In use, when the device 510 is disposed in the collapsed state shown in FIG. 6A, the first support member 540 and the second support member 550 may be folded against the base member 520. In some embodiments, the traverse members (arches) 546, 556 of the first support member 540 and the second support member 550 may be disposed adjacent to the transverse member 526 in the collapsed state.

In the collapsed state, the device 510 may be easily moved, for example, by a single person to a desired location. In the collapsed state as shown in FIG. 6A, the device 510 may be positioned for the procedure. For example, the device 510 may be positioned so that the base member 520 surrounds the patient's head as shown in FIG. 5. For example, the side members 522 and 524 be disposed next to the sides of the patient's head and the traverse member 526 may be disposed near the top of the patient's head. In some embodiments, the base member 520 may be stabilized under the patient's shoulders. The device 510 may also be moved to be at different positions along a patient's body. For example, the device 510 may be positioned at one side of the patient so that the base member is disposed under or adjacent to the side of the patient, positioned at the feet of the patient, among others, or any combination thereof.

After the base member 520 is positioned, the user may then “pop-up” the enclosure 570 by moving the frame 530 to its expanded state. As shown in FIG. 6B, the first support member 540 and the second support member 550 may be moved away from the transverse member 526 so that they are vertical. By moving the second support member 550 away, the second support member 550 may be pivot on the connecting member(s) (hinge) 580 until it is fixed in the vertical state. After which, the first support member 540 may be moved so that the first end/side 542 and the second end/side 544 may move towards the transverse member 526 and fixed in place, for example, using a hole and peg mechanism. The frame 530 may be considered to be in the expanded position when the first support member 540 and the second support member 550 are fixed with respect to the base member 520, as shown in FIG. 6C.

In some embodiments, the devices may further include one or more stabilizing members. For example, the device 510 may include one or more stabilizing members (e.g., a ties, a locking bar, etc.) configured to be disposed on and temporarily join the traverse members (arches 546 and 556), thereby further stabilizing the frame 520 in the expanded state.

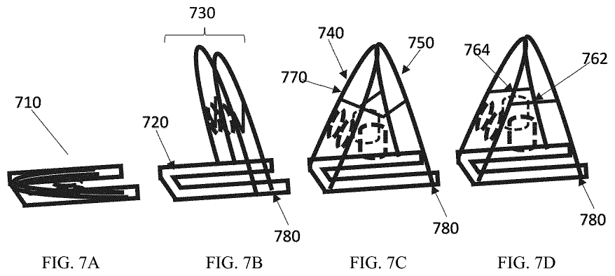

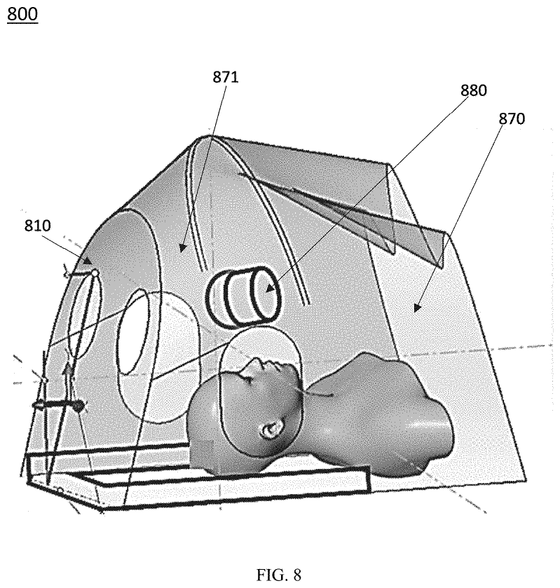

In some embodiments, the device 510 may include one or more stabilizing members positioned between the support members 540 and 550, for example, like as shown in FIG. 4. FIG. 7A-7D shows an example of a device 710 that includes one or more stabilizing members moving between the collapsed state to the expanded state. The device 710 is similar to the device 510 but also includes stabilizing members 762 and 764 disposed between the first supporting member 740 and the second support member 750.

FIG. 7A shows the device 710 in the collapsed state; FIG. 7B shows the device 710 after being positioned and being moved to the expanded state; FIG. 7C shows the device 710 in the expanded state; and FIG. 7 shows the device 710 in the stabilized, expanded state.

After the base member 720 is positioned (FIG. 7A), the user may then “pop-up” the enclosure 770 by moving the frame 730 to its expanded state. As shown in FIG. 7B, the first support member 740 and the second support member 750 may be moved away from the transverse member 726 so that they are vertical. By moving the second support member 750 away, the second support member 750 may be configured to pivot/move outward via a connecting member (e.g., hinge) 780 until it is fixed in the vertical state. After which, the first support member 740 may be moved so that the first end/side 742 and the second end/side 744 may move towards the transverse member 726 and fixed in place, for example, using a hole and peg mechanism as shown in FIG. 7C.

After which, the one or more stabilizing members 762 and 764 may be locked so that the frame 730 is stabilized in its expanded state as shown in FIG. 7D by moving each first portion and each second portion of the members 762, 764 from a folded state shown in FIG. 7C to the locked state shown in FIG. 7D so that the first portion and the second portion form an elongated member. The frame 730 may be considered to be in the expanded position when the first support member 740 and the second support member 750 are fixed with respect to the base member 720 and stabilized, as shown in FIG. 7D.

After use, the device 710 may be decontaminated, for example, by removing particulates using suction or negative pressure, before being collapsed as shown in FIG. 7A.

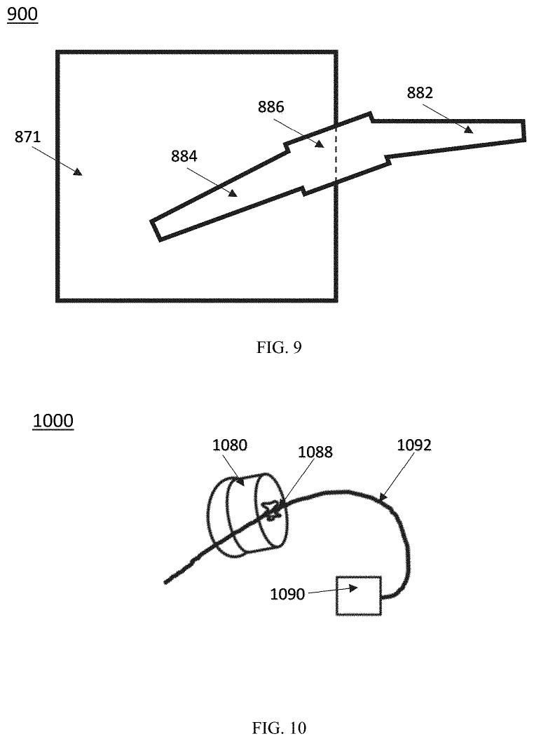

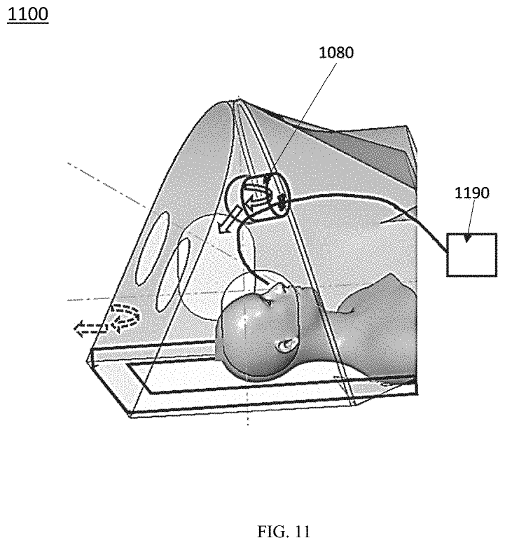

In some embodiments, the infection devices may further include one or more access members that may be air-locked. FIG. 8 shows an example 800 of a device 810 that includes an access member 880 that is configured to be airlocked. FIG. 9 shows a partial enlarged view of the access member 880. As shown in FIG. 9, the access member 880 may include an external wall port 882, an internal wall port 884, and an antechamber 886 disposed therebetween. In use, a device may be placed through the internal wall port 884 and the antechamber 886 and sealed before it enters an enclosure space 871 defined by an enclosure member 870. For example, the access member 880 may include ties or other members at the external wall port 882 to removably surround the instrument. FIG. 10 shows an example 1000 of an access member 1080 in which the external wall port is sealed around wiring or tubing 1092 connected to an instrument 1090 using sealing members 1088.

After the internal wall port 884 is sealed, the instrument may enter the internal port 882 to the enclosure space 871. An airlocked access member can permit free movement of the external technology (e.g., endoscope) while minimizing the risk of aerosol contaminants escaping the enclosed space/enclosure defined by the enclosure member 870.

In some embodiments, one or more airflow devices may be connected to the devices. In some embodiments, the one or more airflow devices may include a pump configured to deliver positive pressure into the airlock antechamber to generate low-level airflow from the antechamber into the main compartment of the containment tent and minimize aerosol leak out of the member. FIG. 11 shows an example 1100 of positive pressure being pumped into the access member 1080 by a pump 1190.

In some embodiments, the one or more access members may include an input valve, for example, for oxygen (e.g., ventilator or bag valve mask). In some embodiments, the one or more access members may include an output valve, for example, to provide negative air flow to evacuate aerosolized particulates during airway management. For example, the output valve can be attached to suction to clear particulates; the output valve can be attached to portal fan to clear particulates; the output valve can also be attached to an incorporated negative pressure waste containment bag. This way, the output valve may be used to evacuate contaminants and minimizing risk to staff prior to disassembling the tent after use.

For example, the negative pressure waste containment bag may be deployed when composite material ribs fixed along the bag are permitted to take their bow shape, opening the bag by mechanical force and creating a negative pressure which draws air and particulates from the enclosure (e.g., tent space) into the containment bag.

In some embodiments, the devices may further include a light, such as LED lights, embedded or attached to the devices to provide light inside the enclosure space.

In some embodiments, the infection devices may be configured to be single-use, disposable devices. In other embodiments, the infection devices may be configured to be sterilizable for multi-use.

The device may be sized for a hospital stretcher, operating room bed, intensive car bed, among others, or any combination thereof.

In some embodiments, the infection control devices may be a part of the system. For example, the system may include the one or more instruments associated with a specific medical procedure to be performed within the protected enclosure in addition to the infection control device. By way of example, the system may include but is not limited to a bag valve mask, laryngoscope, airway kit, an endotracheal tube, a device for securing endotracheal tube, device for confirming endotracheal tube placement, built-in suction tubing port/extension, built-in extension for ventilator circuitry, among others, or any combination thereof.

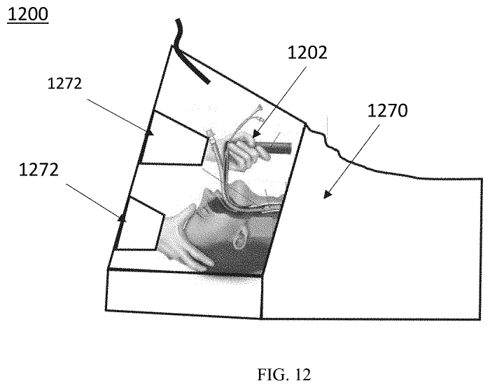

FIG. 12 shows a side view of an example 1200 of a device 1210 being used for an airway management procedure, such as an intubation procedure. In this example, the device 1210 is similar to the device 310 but the device may be according to any of the examples shown and described with respect to the figures. As shown in FIG. 12, a clinician has placed their arms through access members 1272 and corresponding sleeves into the enclosed space/enclosure defined by the enclosure member 1270 to access a patient 1202 for the intubation procedure

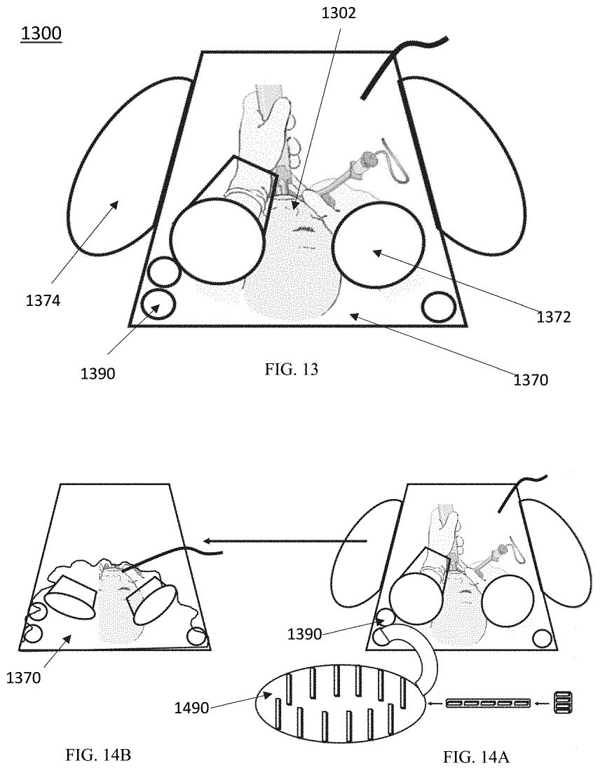

FIG. 13 shows a front view of an example 1300 of a device 1310 being used for an airway management procedure, such as an intubation procedure. In this example, the device 1310 is similar to 1210 but the device may be according to any of the examples shown and described with respect to the figures. Like the device 1210, the device 1310 can include access members 1372, which are sleeves for the clinician. The device 1310 may also include access members 1374 disposed on the side for additional access to the patient 1302 and additional access members 1390 for connection to an external instrument or device, such as one or more air flow devices. For example, the access members 1390 may be ports, such as those of FIGS. 8-11.

For example, as shown in FIG. 14A, after the device 1310 is no longer needed, for example, the patient 1302 no longer needs airway management, the device 1310 may be prepared for removal. To prepare for removal, an evacuation procedure may be performed to remove any contaminants within the enclosed space/enclosure defined by the enclosure member thereby minimizing risk to the clinical space when the device 1310 is removed and disposed of. For example, as shown in FIG. 14A, a containment bag 1490 may be deployed and connected to the enclosed member 1370 to access the enclosed space/enclosure via one or more of the access members 1390. As shown, the bag 1490 may be folded and unfolded for attachment to the device 1310. The bag 1490 may include one or more internal ribs. When attached to the device 1310, the bag 1490 may be configured to mechanically inflate due to these ribs and create negative pressure. After a period of time, the bag 1490 containing any containments or particulates collected from the enclosure defined by the device 1310 may be removed. After the bag 1490 and any pressure is removed, the device 1310 may begin to collapse as shown in FIG. 14B. For example, the enclosure member 1370 may begin to collapse with respect to the frame.

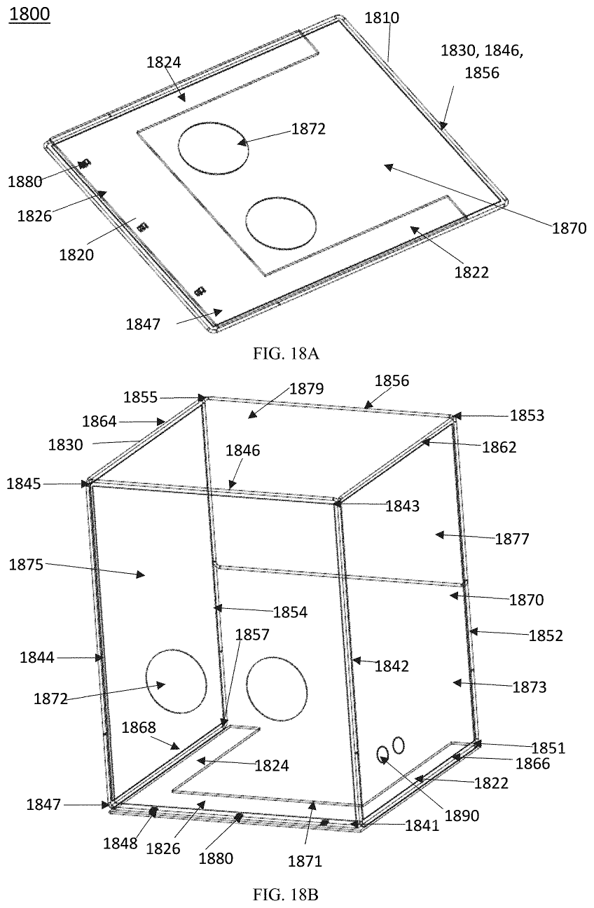

In some embodiments, the frame of the devices may have a different shape, configuration, position with respect to the base member, among others, or a combination thereof. For example, the devices may include a frame having a triangle-shaped side view when expanded, for example, as shown in the FIGS. 15A-17B; a frame including support members having an elongated, straight traverse members, as shown in FIGS. 15A-18B; a frame having a square or rectangular-shaped side view when expanded as shown in FIGS. 18A-B; among others; or any combination thereof.

FIGS. 15A and 15B show an example 1500 of a device 1510 having a first support member 1540 and a second support member 1550. FIG. 15A shows the device 1510 in a collapsed state and FIG. 15B shows the device 1510 in an expanded state. In some embodiments, like the devices shown and described with respect to FIGS. 1-14B, the device 1510 may include a base member 1520 having a U-shape. The base member 1520 may include a first side member 1522, a second side member 1524 parallel to the first side member 1522, and a traverse member 1526 that is disposed between the first side member 1522 and the second side member 1524.

In some embodiments, the frame 1530 may include a first support member 1540 and a second support member 1550. In some embodiments, the first support member 1540 may include a first side 1542 and a second side 1544 parallel to the first side 1542. The first side 1542 may have a first end 1541, a second end 1543, and a length therebetween; and the second side may have a first end 1545, a second end 1547, and a length therebetween. In some embodiments, the first support member 1540 may include a first traverse member 1546 disposed between the first ends 1541 and 1545 and a second traverse member 1548 parallel to the first traverse member 1546. The second traverse member 1548 may be disposed between the second ends 1543 and 1547. In some embodiments, the second traverse member 1548 may be disposed so as to extend along the traverse member 1526 of the base member 1520.

In some embodiments, the second support member 1550 may include a first side 1552 and a second side 1554 parallel to the first side 1552. The first side 1552 may have a first end 1551, a second end 1553, and a length therebetween; and the second side may have a first end 1555, a second end 1557, and a length therebetween. In some embodiments, the second support member 1550 may include a traverse member 1556 disposed between the first ends 1541 and 1545.

In some embodiments, the traverse members 1546, 1556 may have an elongated, straight shape. In other embodiments, the traverse members 1546, 1556 may have a different shape, such as an arch, for example, as shown in and described with respect to FIGS. 1-11.

In some embodiments, the first traverse member 1546 and the second traverse member 1556 may be integrated so as to form a single traverse member, may be joined or coupled to each other, among others, or a combination thereof.

In some embodiments, like the enclosure members of the other figures, the enclosure member 1570 may be disposed on the frame 1530 and the base member 1520. For example, the enclosure member 1570 may be coupled to the base member 1520 and the first support member 1540 and the second support member 1550.

In some embodiments, the enclosure member 1570 may include a plurality of panels of enclosure. In this example, the enclosure member 1570 may include four panels 1571, 1573, 1575, and 1577. For example, a panel 1571 may be disposed so as to be coupled to and between (i) the traverse member 1546, 1556 and the traverse member 1548 and (ii) the first and second sides 1542 and 1544; a panel 1573 may be disposed so as to be coupled to and between (i) the traverse member 1546, 1556, (ii) the sides 1542 and 1552, and (ii) the side member 1522 of the base member 1520; a panel 1575 may be disposed so as to be coupled to and between (i) the traverse member 1546, 1556, (ii) the sides 1544 and 1554, and (iii) the side member 1524 of the base member 1520; and a panel 1577 may be disposed so as to be coupled to the traverse member 1546, 1556. The panel 1577 may be disposed so as to be only coupled to the traverse member 1546, 1556 and be disposed between the sides 1552 and 1554. This way, the panel 1577 can be freely moved while the other panels can create a seal minimizing aerosol escape from the panel 1577 into the vicinity of clinical team members treating the patient in that space. In some embodiments, the panel 1577 may also be at least partially coupled to the sides 1552 and 1554.

In some embodiments, like the access members of the other figures, the device 1510 may include at least one set of access members 1572 disposed within the enclosure member 1570 and configured to provide a set of user's hands access to the enclosed space/enclosure defined by the enclosure member 1570. For example, the access members 1572 may be sleeves configured for the clinician/staff's arms/hands. In some embodiments, the device 1510 may include one or more access members 1590, such as ports, configured to connect to other devices, such as a negative pressure device. For example, the one or more access ports 1590 may include but is not limited to those described in FIGS. 11-14B.

In some embodiments, the first support member 1540 and the second support member 1550 may be rotationally coupled to the base member 1520 by one or more connecting members 1580. For example, the one or more connecting members 1580 may include one or more mechanical connecting member(s) (e.g., pins, hinges, other mechanical fasteners, etc.); a portion of the enclosure member 1570; other fasteners (e.g., an adhesive); the frame 1530 itself (e.g., partially integrated (e.g., at the respective traverse members)); among others; or a combination thereof.

In some embodiments, the one or more connecting members 1580 may include one or more hinges 1582 disposed between the base member 1520 and the first support member 1540. For example, the one or more hinges 1582 may be disposed along the length and between the traverse member 1526 of the base member 1520 and the traverse member 1546 of the first support member 1540.

In some examples, the second support member 1550 may be rotationally coupled to the base member 1520 by a set of one or more connecting members 1584. For example, the one or more connecting members 1584 may be pins. For example, each side 1552, 1554 may be coupled to the respective side member 1522, 1524 of the base member 1520 via a pin 1584 disposed between each of the ends 1553, 1557 and side members 1522, 1524, respectively. In other embodiments, different connecting members 1580 may be used to couple the first support member 1540 and the second support member 1550 to the frame 1520.

In some embodiments, the device 1510 may include one or more stabilizing members 1560 configured to stabilize the first support member 1520 when in an expanded position. In this example, the stabilizing member 1560 may be a locking bar rotatably coupled to the first support member 1540 via connecting members 1561 and 1563. For example, the connecting members 1561 and 1563 may be pins. In some embodiments, the stabilizing member 1560 may include a first side 1562 and a second side 1564 respectively coupled to the first side 1542 and the second side 1543, respectively, and extending therefrom. The stabilizing member 1560 may include a traverse member 1566 disposed between the sides 1562 and 1564 and configured to be disposed on the base member 1520 and in between the first support member 1540 and the second support member 1550 when the frame 1530 is in expanded state.

In some embodiments, the base member 1520 may include one or more complimentary receiving members 1528 to receive the traverse member 1566 of the stabilizing member 1560, such as detents, grooves, notches, among others, or a combination thereof. In the example, the receiving member(s) 1528 may be disposed on the base member 1520 between the first support member 1530 and the second side member 1540 when the frame 1530 is expanded. When the traverse member 1566 is disposed in the receiving member 1528, the frame 1530 may be (temporarily) locked in the expanded state.

When the device 1510 is disposed in the collapsed state shown in FIG. 15A, for example, for storage before and/or after use, the first support member 1540, the enclosure member 1570, and the stabilizing member 1560 may be folded against the base member 1520. In some embodiments, the traverse member 1546 may be disposed beyond the base member 1520 in the collapsed state.

When the device 1510 is ready to be used, the device 1510 may be expanded by moving the frame 1530 to its expanded state. As shown in FIG. 15B, the first support member 1540 and the second support member 1550 may be moved vertically by moving the traverse member 1546, 1556 toward the transverse member 1526. By moving the support members 1540 and 1550 vertically, the first support member 1540 may pivot/rotate via the hinge(s) 1582 and the second support member 1550 may be pivot/rotate via the pins 1584, respectively until the frame is in the vertical state. After which, if necessary, the stabilizing member 1560 may be moved so that the traverse member 1566 sits/rests in the receiving member 1528 of the base member 1520 so as to lock the stabilizing member 1566. The frame 1530 may be considered to be in the expanded position when the support members 1540 and 1550 are stabilized with respect to the base member 1520 by the “locked” stabilizing member 1560, for example, as shown in FIG. 15B.

In the collapsed state, the device 1510 may be easily moved, for example, by a single person to a desired location. In the collapsed state as shown in FIG. 15A or expanded state shown in FIG. 15B, the device 1510 may be positioned for the procedure. For example, the device 1510 may be positioned so that the base member 1520 is disposed to surround on the patient's head. In some embodiments, the base member 1520 may be stabilized under the patient's shoulders. The device 1510 may also be moved to be at different positions along a patient's body. For example, the device 1510 may be positioned at one side of the patient so that the base member 1520 is disposed under or adjacent to the side of the patient, positioned at the feet of the patient, among others, or any combination thereof.

In some embodiments, the devices may include a stabilizing member that is configured to be closer to the ends of the side members of the base member so as to be disposed within the frame (interior of the frame) when the frame is in the expanded state. For example, as shown in FIGS. 15A and 15B, the stabilizing member 1560 may be disposed between the first support member 1540 and the second support member 1550. In some embodiments, the stabilizing member may be disposed outside the frame.

FIGS. 16A and 16B show an example 1600 of a device 1610 having a stabilizing member 1660 that is configured to be disposed on the base member exterior to the frame 1630 when the frame is in the expanded state. FIG. 16A shows an example 1600 of the device 1610 in a collapsed state and 16B shows the device 1610 in an expanded state. In some embodiments, like the devices shown and described with respect to FIGS. 1-15B, the device 1610 may include a base member 1620 having a U-shape. The base member 1620 may include a first side member 1622, a second side member 1624 parallel to the first side member 1622, and a traverse member 1626 that is disposed between the first side member 1622 and the second side member 1624.

In some embodiments, like the frame 1530, the frame 1630 may include a first support member 1640 and a second support member 1650. In some embodiments, the first support member 1640 may include a first side 1642 and a second side 1644 parallel to the first side 1642. The first side 1642 may have a first end 1641, a second end 1643, and a length therebetween; and the second side may have a first end 1645, a second end 1647, and a length therebetween. In some embodiments, the first support member 1640 may include a first traverse member 1646 disposed between the first ends 1641 and 1645 and a second traverse member 1648 parallel to the first traverse member 1646. The second traverse member 1648 may be disposed between the second ends 1643 and 1647. In some embodiments, the second traverse member 1648 may be disposed so as to extend along the traverse member 1626 of the base member 1620.

In some embodiments, the second support member 1650 may include a first side 1652 and a second side 1654 parallel to the first side 1652. The first side 1652 may have a first end 1651, a second end 1653, and a length therebetween; and the second side may have a first end 1655, a second end 1657, and a length therebetween. In some embodiments, the second support member 1650 may include a traverse member 1656 disposed between the first ends 1641 and 1645.

In some embodiments, the traverse members 1646, 1656 may have an elongated, straight shape. In other embodiments, the traverse members 1646, 1656 may have a different shape, such as an arch, for example, as shown in and described with respect to FIGS. 1-11.

In some embodiments, like the frame 1530, the first traverse member 1646 and the second traverse member 1656 may be integrated so as to form a single traverse member; may be joined to or coupled to each other; among others; or any combination thereof

In some embodiments, like the enclosure members of the other figures, the enclosure member 1670 may be disposed on the frame 1630 and the base member 1620. For example, the enclosure member 1670 may be coupled to the base member 1620 and the first support member 1640 and the second support member 1650.

In some embodiments, like the enclosure member 1570, the enclosure member 1670 may include a plurality of panels of enclosure. In this example, the enclosure member 1670 may include four panels 1671, 1673, 1675, and 1677. For example, a panel 1671 may be disposed so as to be coupled to and between (i) the traverse member 1646, 1656 and the traverse member 1648 and (ii) the first and second sides 1642 and 1644; a panel 1673 may be disposed so as to be coupled to and between (i) the traverse member 1646, 1656, (ii) the sides 1642 and 1652, and (iii) the side member 1622 of the base member 1620; a panel 1675 may be disposed so as to be coupled to and between (i) the traverse member 1646, 1656, (ii) the sides 1644 and 1654, and (iii) the side member 1624 of the base member 1620; and a panel 1677 may be disposed so as to be coupled to the traverse member 1646, 1656. In some embodiments, the panel 1677 may be disposed so as to be only coupled to the traverse member 1646, 1656 and be disposed between the sides 1652 and 1654. This way, the panel 1677 can be freely moved while the other panels can create a seal minimizing aerosol escape from the panel 1677 into the vicinity of clinical team members treating the patient in that space. In some embodiments, the panel 1677 may also be at least partially d to the sides 1652 and 1654.

In some embodiments, like the access members of the other figures, the device 1610 may include at least one set of access members 1672 disposed within the enclosure member 1670 and configured to provide a set of user's hands access to the enclosed space/enclosure defined by the enclosure member 1670. For example, the access members 1672 may be sleeves configured for the clinician/staff's arms/hands. In some embodiments, the one or more access members 1690, such as ports, configured to connect to other devices, such as a negative pressure device. For example, the one or more access ports 1690 may be similar to those shown in FIGS. 13-14B.

In some embodiments, the first support member 1640 and the second support member 1650 may be rotationally coupled to the base member 1620 by one or more connecting members 1680. For example, the one or more connecting members 1680 may include one or more mechanical connecting member(s) (e.g., pins, hinges, other mechanical fasteners, etc.); a portion of the enclosure member 1670; other fasteners (e.g., an adhesive); the frame 1630 itself (e.g., partially integrated (e.g., at the respective traverse members)); among others; or a combination thereof.

In some embodiments, the one or more connecting members 1680 may include a one or more connecting members 1684. For example, the one or more connecting members 1684 may be pins. By way of example, each side 1642, 1644 may be coupled to the respective side member 1622, 1624 of the base member 1620 via a pin 1684 disposed between each of the ends 1643, 1647 and side members 1622, 1624, respectively; and each side 1652, 1654 may be coupled to the respective side member 1622, 1624 of the base member 1620 via a pin 1684 disposed between each of the ends 1653, 1657 and side members 1622, 1624, respectively. In other embodiments, different connecting members 1680 may be used to rotatably couple the first support member 1640 and the second support member 1650 to the base member 1620.

In some embodiments, the device 1610 may include one or more stabilizing members 1660 configured to stabilize the first support member 1620 when in an expanded position. In this example, the stabilizing member 1660 may be a locking bar rotatably coupled to the first support member 1640 via connecting members 1661 and 1663. For example, the connecting members 1661 and 1663 may be pins. In some embodiments, the stabilizing member 1660 may include a first side 1662 and a second side 1664 respectively coupled to the first side 1642 and the second side 1643, respectively, and extending therefrom. The stabilizing member 1660 may include a traverse member 1666 disposed between the sides 1662 and 1664 and configured to be disposed on the base member 1620 exterior to the support member 1640 when the frame 1630 is in expanded state. In some embodiments, the base member 1620 may include one or more complimentary receiving members 1628 to receive the traverse member 1666 of the stabilizing member 1660, such as detents, grooves, notches, among others, or a combination thereof. In the example, the receiving member(s) 1628 may be disposed on the traverse member 1626 between the first support member 1640 and the side end of the base member 1620 when the frame 1630 is expanded. When the traverse member 1666 is disposed in the receiving member 1628, the frame 1630 may be (temporarily) locked in the expanded state.

When the device 1610 is disposed in the collapsed state shown in FIG. 16A, for example, for storage before and/or after use, the first support member 1640, the enclosure member 1670, and the stabilizing member 1660 may be folded against the base member 1620. In some embodiments, the traverse member 1646 may be disposed beyond the base member 1620 in the collapsed state.

When the device 1610 is ready to be used, the device 1610 may be expanded by moving the frame 1630 to its expanded state. As shown in FIG. 16B, the first support member 1640 and the second support member 1650 may be moved vertically by pulling the stabilizing member 1660. By pulling the stabilizing member 1660, the first support member 1640 and the second support members 1650 may pivot/rotate via the connecting members 1684, respectively until the frame 1630 is in the vertical state. After which, if necessary, the stabilizing member 1660 may be moved so that the traverse member 1666 sits/rests in the receiving member(s) 1628 of the base member 1620 so as to lock the stabilizing member 1666. The frame 1630 may be considered to be in the expanded position when the support members 1640 and 1650 are stabilized with respect to the base member 1620 by the “locked” stabilizing member 1660, for example, as shown in FIG. 16B.

In the collapsed state, the device 1610 may be easily moved, for example, by a single person to a desired location. In the collapsed state as shown in FIG. 16A or expanded state shown in FIG. 16B, the device 1610 may be positioned for the procedure. For example, the device 1610 may be positioned so that the base member 1620 is surrounds the patient's head. In some embodiments, the base member 1620 may be stabilized under the patient's shoulders. The device 1610 may also be moved to be at different positions along a patient's body. For example, the device 1610 may be positioned at one side of the patient so that the base member 1620 is disposed under or adjacent to the side of the patient, positioned at the feet of the patient, among others, or any combination thereof.

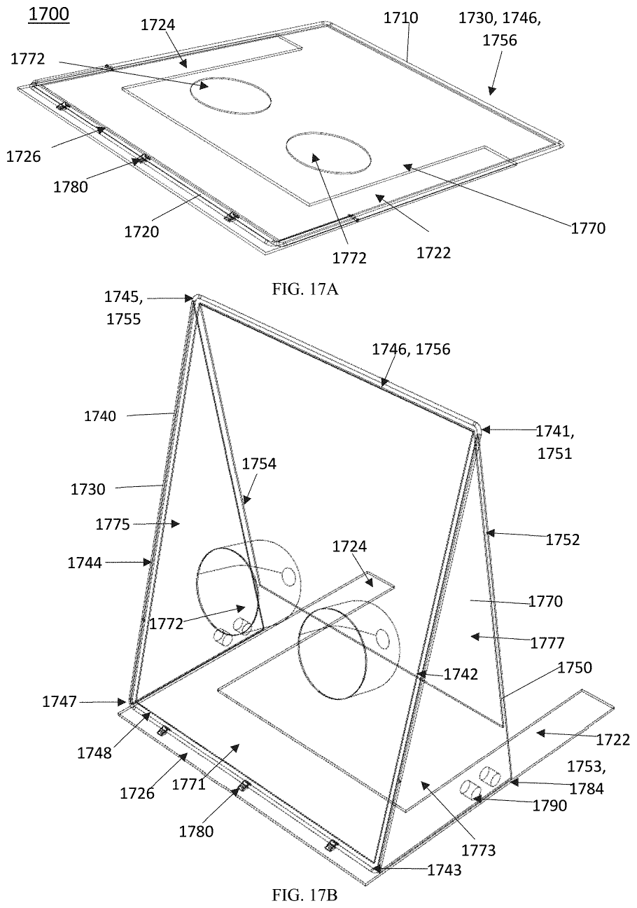

In some embodiments, the devices omit a stabilizing member to stabilize (temporarily lock) the frame in the expanded state. FIGS. 17A and 17B show an example 1700 of a device 1710 including a frame 1730 that is configured to be tensioned to be in the expanded stated and/or folded/collapsed state with respect to a base member 1720. FIG. 17A shows the device 1710 in a collapsed state and 17B shows the device 1710 in an expanded state.

In some embodiments, like the devices shown and described with respect to FIGS. 1-16B, the device 1710 may include a base member 1720 having a U-shape. The base member 1720 may include a first side member 1722, a second side member 1724 parallel to the first side member 1722, and a traverse member 1726 that is disposed between the first side member 1722 and the second side member 1724.

In some embodiments, like the frames 1530 and 1630, the frame 1730 may include a first support member 1740 and a second support member 1750. In some embodiments, the first support member 1740 may include a first side 1742 and a second side 1744 parallel to the first side 1742. The first side 1742 may have a first end 1741, a second end 1743, and a length therebetween; and the second side 1744 may have a first end 1745 and a second end 1747. In some embodiments, the first support member 1740 may include a first traverse member 1746 disposed between the first ends 1741 and 1745 and a second traverse member 1748 parallel to the first traverse member 1746. The second traverse member 1748 may be disposed between the second ends 1743 and 1747. In some embodiments, the second traverse member 1748 may be disposed so as to extend along the traverse member 1726 of the base member 1720.

In some embodiments, the second support member 1750 may include a first side 1752 and a second side 1754 parallel to the first side 1752. The first side 1752 may have a first end 1751, a second end 1773, and a length therebetween; and the second side may have a first end 1755, a second end 1777, and a length therebetween. In some embodiments, the second support member 1750 may include a traverse member 1776 disposed between the first ends 1741 and 1745.

In some embodiments, the traverse members 1746, 1756 may have an elongated, straight shape. In other embodiments, the traverse member 1746, 1756 may have a different shape, such as an arch, for example, as shown in and described with respect to FIGS. 1-11.

In some embodiments, like the enclosure members of the other figures, the enclosure member 1770 may be disposed on the frame 1730 and the base member 1720. For example, the enclosure member 1770 may be coupled to the base member 1720 and the first support member 1740 and the second support member 1750.

In some embodiments, the enclosure member 1770 may include a plurality of panels of enclosure. In this example, like FIGS. 15 and 16, the enclosure member 1770 may include four panels 1771, 1773, 1775, and 1777. For example, a (enclosure) panel 1771 may be disposed so as to be coupled to and between (i) the traverse member 1746, 1756 and the traverse member 1748 and (ii) the first and second sides 1742 and 1744; a panel 1773 may be disposed so as to be coupled to and between (i) the traverse member 1746, 1756, (ii) the sides 1742 and 1752, and (iii) the side member 1722 of the base member 1720; a panel 1775 may be disposed so as to be coupled to and between (i) the traverse member 1746, 1756, (ii) the sides 1744 and 1754, and (iii) the side member 1724 of the base member 1720; and a panel 1777 may be disposed so as to be coupled to the traverse member 1746, 1756. The panel 1777 may be disposed so as to be only coupled to the traverse member 1746, 1756 and be disposed between the sides 1752 and 1754. This way, the panel 1777 can be freely moved while the other panels can create a seal minimizing aerosol escape from the panel 1777 into the vicinity of clinical team members treating the patient in that space. In some embodiments, the panel 1777 may also be at least partially coupled to the sides 1752 and 1754.

In some embodiments, like the access members of the other figures, the device 1710 may include at least one set of access members 1772 disposed within the enclosure member 1770 and configured to provide a set of user's hands access to the enclosed space/enclosure defined by the enclosure member 1770. For example, the access members 1772 may be sleeves configured for the clinician/staff's arms/hands. In some embodiments, the device 1710 may include one or more access members 1790, such as ports, configured to connect to other devices, such as a negative pressure device. For example, the one or more access ports 1790 may be similar to those shown in FIGS. 11-14B.

In some embodiments, the first support member 1740 and the second support member 1750 may be rotationally coupled to the base member 1720 by one or more connecting members 1780. For example, the one or more connecting members 1780 may include one or more mechanical connecting member(s) (e.g., pins, hinges, other mechanical fasteners, etc.), a portion of the enclosure member; other fasteners (e.g., an adhesive); the frame 1730 itself (e.g., partially integrated (e.g., at the respective traverse members)); among others; or a combination thereof.

For example, the one or more connecting members 1780 may include one or more connecting members 1782 disposed between the base member 1720 and the first support member 1740. By way of example, the one or more connecting members 1782 may be hinges. In some embodiments, the one or more hinges 1782 may be disposed along the length and between the traverse member 1726 of the base member 1720 and the traverse member 1746 of the first support member 1740.

In some examples, the second support member 1750 may be rotationally coupled to the base member 1720 by a set of connecting members 1784. For example, the set of connecting members 1784 may be pins. For example, each side 1752, 1754 may be coupled to the respective side member 1722, 1724 of the base member 1720 via a pin 1784 disposed between each of the ends 1753, 1757 and side members 1722, 1724, respectively. In other embodiments, different connecting members 1780 may be used to rotatably couple the first support member 1740 and the second support member 1750 to the base member 1720.

In some embodiments, the frame 1730 may be configured to be tensioned to be in the expanded state with respect to the base member 1720. For example, the first support member 1740 and/or the second support member 1750 may be tensioned to be in a vertical position (substantially perpendicular to) the base member 1720. In some embodiments, the frame 1730 may be disposed in the collapsed/folded state as shown in FIG. 17A when the frame 1730 is removably attached to the base member 1720, for example, using a fastener, such as a strap, loop, clip, among others, or a combination thereof. This way, the frame 1730 can moved from a forced folded/collapsed state, for example, when the fastener is removed/unattached with respect to the base member 1720 and the frame 1730, and to the expanded state.

In some embodiments, the frame 1730 may be configured to be tensioned to be in the folded or collapsed state with respect to the base member 1720. For example, the first support member 1740 and/or the second support member 1750 may be tensioned to be disposed directly on the base member 1720 in the folded state. In this position, the support members 1740, 1750 may be substantially parallel to the base member 1720. This way, the frame 1730 can moved from the folded/collapsed state to the expanded state when a user forces the frame 1730 to the vertical position (towards the traverse member 1726).

When the device 1710 is disposed in the collapsed state shown in FIG. 17A, for example, for storage before and/or after use, the first support member 1740, the enclosure member 1770, and the stabilizing member 1760 may be folded against the base member 1720. In some embodiments, the device 1710 may further include a fastener disposed around the frame 1730 and the base member 1720 so as to be disposed around the device 1710 including the traverse members 1746, 1756 and traverse member 1726. In some embodiments, the traverse member 1746 may be disposed beyond the base member 1720 in the collapsed state.

When the device 1710 is ready to be used, the device 1710 may be expanded by moving the frame 1730 to its expanded state by removing the fastener or by using force. As shown in FIG. 17B, if by force, the first support member 1740 and the second support member 1750 may be moved vertically by moving the traverse member 1746, 1756 toward the transverse member 1726. By moving the support members 1740 and 1750 vertically, the first support member 1740 and the second support members 1750 may pivot/rotate via the hinges 1782 and the pins 1784, respectively until the frame 1730 is in the vertical state. The frame 1730 may be considered to be in the expanded position when the frame 1730 is vertical, for example, as shown in FIG. 17B.

In the collapsed state, the device 1710 may be easily moved, for example, by a single person to a desired location. In the collapsed state as shown in FIG. 17A or expanded state shown in FIG. 17B, the device 1710 may be positioned for the procedure. For example, the device 1710 may be positioned so that the base member 1720 surrounds a head of the patient. In some embodiments, the base member 1720 may be stabilized under the patient's shoulders. The device 1710 may also be moved to be at different positions along a patient's body. For example, the device 1710 may be positioned at one side of the patient so that the base member 1720 is disposed under or adjacent to the side of the patient, positioned at the feet of the patient, among others, or any combination thereof.

In some embodiments, the frame of the device may have a different shape. For example, the frame may have a square-like side view when expanded. FIGS. 18A and 18B show an example 1800 of a device 1810 including a frame 1830 that a square side view when in the expanded state with respect to a base member 1820. FIG. 18A shows the device 1810 in a collapsed state and 18B shows the device 1810 in an expanded state.

In some embodiments, like the devices shown and described with respect to FIGS. 1-17B, the device 1810 may include a base member 1820 having a U-shape. The base member 1820 may include a first side member 1822, a second side member 1824 parallel to the first side member 1822, and a traverse member 1826 that is disposed between the first side member 1822 and the second side member 1824.

In some embodiments, the frame 1830 may include a first support member 1840 and a second support member 1850. In some embodiments, the first support member 1840 may include a first side 1842 and a second side 1844 parallel to the first side 1842. The first side 1842 may have a first end 1841, a second end 1843, and a length therebetween; and the second side 1844 may have a first end 1845 and a second end 1847. In some embodiments, the first support member 1840 may include a first traverse member 1846 disposed between the first ends 1841 and 1845 and a second traverse member 1848 parallel to the first traverse member 1846. The second traverse member 1848 may be disposed between the second ends 1843 and 1847. In some embodiments, the second traverse member 1848 may be disposed so as to extend along the traverse member 1826 of the base member 1820.

In some embodiments, the second support member 1850 may include a first side 1852 and a second side 1854 parallel to the first side 1852. The first side 1852 may have a first end 1851, a second end 1873, and a length therebetween; and the second side may have a first end 1855, a second end 1877, and a length therebetween. In some embodiments, the second support member 1850 may include a traverse member 1876 disposed between the first ends 1841 and 1845.

In some embodiments, the traverse members 1846, 1856 may have an elongated, straight shape. In other embodiments, the traverse member 1846, 1856 may have a different shape, such as an arch, for example, as shown in and described with respect to FIGS. 1-11.

In some embodiments, the device 1810 may include a first (frame) traverse member 1862 and a second (frame) traverse member 1864 that is disposed between and couples the first support member 1840 and the second support member 1840. In some embodiments, the first traverse member 1862 may be disposed between the ends 1843 and 1853, and the second traverse member 1864 may be disposed between the ends 1845 and 1853. When the frame 1830 is in an expanded state, the first traverse member 1862 may be parallel to the first side member 1822 and the second traverse member 1864 may be parallel to the second side member 1824.

In some embodiments, the frame 1830 may also include a third (frame) traverse member 1866 disposed between the ends 1841 and 1851, and a fourth (frame) traverse member 1868 disposed between the ends 1847 and 1857. In other embodiments, the (frame) traverse members 1866 and/or 1868 may be omitted.

In some embodiments, like the other devices, the device 1810 may include an enclosure member 1870 may be disposed on the frame 1830 and between the base member 1820. For example, the enclosure member 1870 may be coupled to and between the base member 1820 and the first support member 1840 and the second support member 1850.

In some embodiments, the enclosure member 1870 may include a plurality of panels of enclosure. In this example, the enclosure member 1870 may include five panels 1871, 1873, 1875, 1877, and 1879. For example, a panel 1871 may be disposed so as to be coupled to and between (i) the traverse member 1846, 1848 and (ii) the first and second sides 1842 and 1844; a panel 1873 may be disposed so as to be coupled to and between (i) the traverse member 1862, (ii) the sides 1842 and 1852, and (iii) the side member 1822 of the base member 1820; a panel 1875 may be disposed so as to be coupled to and between (i) the traverse member 1864, (ii) the sides 1844 and 1854, and (iii) the side member 1824 of the base member 1820; a panel 1877 may be disposed so as to be coupled to and between the traverse member 1856; and a panel 1879 may be disposed so as to be coupled to and between (i) the traverse members 1846 and 1856, and (ii) the traverse members 1862 and 1864. In some embodiments, the panel 1877 may only be directly coupled to the traverse member 1856 and be disposed between the sides 1852 and 1854. This way, the panel 1877 can be freely moved while the other panels can create a seal minimizing aerosol escape from the panel 1877 into the vicinity of clinical team members treating the patient in that space. In some embodiments, the panel 1877 may also be at least partially coupled to the sides 1852 and 1854.

In some embodiments, like the access members of the other figures, the device 1810 may include at least one set of access members 1872 disposed on the enclosure member 1870 and configured to provide a set of user's hands access to the enclosed space/enclosure defined by the enclosure member 1870. For example, the access members 1872 may be sleeves configured for the clinician/staff's arms/hands. In some embodiments, the device 1810 may include one or more access members 1890, such as access ports configured to connect to other devices, such as a negative pressure device. For example, the one or more access ports may include but are not limited to those described in FIGS. 11-14B.

In some embodiments, the first support member 1840 and/or the second support member 1850 may be rotationally coupled the base member 1820 by one or more connecting members 1880. For example, the one or more connecting members 1880 may include one or more mechanical connecting member(s) (e.g., pins, hinges, other mechanical fasteners, etc.); a portion of the enclosure member; other fasteners (e.g., an adhesive); the frame 1830 itself (e.g., partially integrated (e.g., at the respective traverse members)); among others; or a combination thereof. By way of example, at least a portion of the traverse member 1848 of the first support member 1830 may be coupled to the base member 1820 by the one or more connecting members 1880.

When the device 1810 is disposed in the collapsed state shown in FIG. 18A, for example, for storage before and/or after use, the panel panels 1871, 1873, 1875, and 1879 may be folded so that the frame may rest against the base member 1820.

When the device 1810 is ready to be used, the device 1810 may be expanded by unfolding the panels of the frame 1830 so it is in its expanded state as shown in FIG. 18B.

In the collapsed state, the device 1810 may be easily moved, for example, by a single person to a desired location. In the collapsed state as shown in FIG. 18A or expanded state shown in FIG. 18B, the device 1810 may be positioned for the procedure. For example, the device 1810 may be positioned so that the base member 1820 surrounds a head of the patient. In some embodiments, the base member 1820 may be stabilized under the patient's shoulders. The device 1810 may also be moved to be at different positions along a patient's body. For example, the device 1810 may be positioned at one side of the patient so that the base member 1820 is disposed under or adjacent to the side of the patient, positioned at the feet of the patient, among others, or any combination thereof.

In some embodiments, the base member may be made of one or more metal materials, such as steel. In some embodiments, the support member(s) and/or stabilizing member may be a wireframe. In some embodiments, the enclosure may be made of one or more transparent materials, such as a tarp-like material. For example, the materials may include synthetic materials, such as nylon, plastic, among others, or a combination thereof.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. An infection control device, comprising:
a base member, the base member having:
a first side member that has first end, a second end, and a length;
a second side member that is parallel to the first side member, the first end having a first end, a second end, and a length; and
a traverse member that has a length and is disposed between the first end of the first side member and the first end of the second side member;
a frame disposed on the base member, the frame including a first support member and a second support member that is spaced from the first support member with respect to the base member;
an enclosure member disposed on the frame and the base member, the enclosure member configured to define an enclosure above the base member; and
one or more access members disposed within the enclosure member and configured to provide access to the enclosure;
wherein the frame is configured to move between a collapsed state and an expanded state with respect to the base member; and
wherein when the frame is in the expanded state, the first support member and the second support member are configured to extend vertically with respect to the base member.

2. The infection control device according to claim 1, wherein the base member has a u-shape.

3. The infection control device according to claim 2, wherein the frame is a wire-frame.

4. The infection control device according to claim 3, further comprising:
one or more stability members disposed on the first support frame and/or the second support frame, the one or more stability members being configured to stabilize the frame in the expanded state.

5. The infection control device according to claim 3, wherein:
the one or more stability members includes:
a first stability member disposed between the first support member and the second support member, the first stability member including a first portion and a second portion; and
a second stability member disposed between the first support member and the second support member, the second stability member including a first portion and second portion;
each of the first stability member and the second stability member is configured to move between (i) a folded state in which the respective first portion and the second portion fold with respect to each other and (ii) a locked state in which the respective first portion and the second portion couples so that the respective first portion and the second portion form a straight elongated member.

6. The infection control device according to claim 3, wherein the first support member is closer to the traverse member of the base member than the second support member.

7. The infection control device according to claim 6, wherein the one or more access members are configured to be coupled to one or more airflow devices, the one or more airflow devices being configured to deliver negative pressure to the enclosure.

8. The infection control device according to claim 7, wherein the one or more air flow devices includes a containment bag configured to deliver negative pressure to the enclosure and collect containments disposed within the enclosure.

9. The infection control device according to claim 7, wherein the one or more access members are air-locked.

10. The infection control device according to claim 6, wherein:
the first support member includes a first side, a second side, and a traverse member therebetween;
the second support member includes a first side, a second side, and a traverse member therebetween; and
ends of the first support member and the ends of the second support member are configured to be disposed on the base member.

11. The infection control device according to claim 10, wherein the traverse member of the first support member and the traverse member of the second support member are spaced apart with respect to the base member.

12. The infection control device according to claim 10, wherein a portion of the traverse member of the first support member and a portion of the traverse member of the second support member are coupled.

13. The infection control device according to claim 10, wherein the first support and the second support member are different sizes.

14. The infection control device according to claim 10, wherein the first support member and the second support member are substantially the same size.

15. The infection control device according to claim 10, wherein each of the traverse member of the first support member and the traverse member of the second support member has an arch shape.

16. The infection control device according to claim 10, wherein each of the traverse member of the first support member and the traverse member of the second support member has an elongated, straight shape.

17. The infection control device according to claim 16, wherein the traverse member of the first support member and the traverse member of the second support member are coupled so that the frame has a triangular side view when in the expanded state.

18. The infection control device according to claim 17, further comprising:
a stabilizing member disposed on the first support member so as to be closer to or further away from the traverse member of the base member;
wherein:
the stabilizing member configured to stabilize the frame in the expanded state; and
the base member includes one or more receiving members configured to receive a portion of the stabilizing member when the frame is in the expanded state.

19. The infection control device according to claim 18, wherein the stabilizing member is a bar.

20. The infection control device according to claim 16, wherein:
the frame further includes:
a first frame traverse member disposed between one end of the traverse member of the first support member and one end of the traverse member of the second support member; and
a first frame traverse member disposed between another end of the traverse member of the first support member and another end of the traverse member of the second support member;
the frame has a rectangular side view when the frame is in the expanded state.
